(19) United States
(12) Patent Application Publication
(10) Pub. No.: US 2022/0296488 A1
(43) Pub. Date: Sep. 22, 2022

(54) COMPOSITIONS COMPRISING AT LEAST ONE COMPOUND OF THE N-ACYLAMINO AMIDE FAMILY AND AT LEAST ONE ALKYL POLYGLYCOSIDE

(71) Applicant: L'OREAL, Paris (FR)

(72) Inventors: Chantal JOUY, Saint-Ouen (FR); Marie-Lise CHIRON, Chevilly Larue (FR); Sandrine THEROUIN-KOELY, Chevilly Larue (FR); Pamella WANG, Chevilly Larue (FR)

(21) Appl. No.: 17/616,036

(22) PCT Filed: Jun. 19, 2020
(86) PCT No.: PCT/EP2020/067220

§ 371 Date: Dec. 2, 2021

(30) Foreign Application Priority Data
Jun. 19, 2019 (FR) FR1906608

(51) Int. Cl. A61K 8/60; A61K 8/06; A61Q 19/00; A61K 8/44

(52) U.S. Cl. CPC A61K 8/604; A61K 8/062; A61Q 19/00; A61K 8/44

(57) ABSTRACT

The invention relates to a composition, in particular a cosmetic composition, preferably in the form of an oil-in-water emulsion, comprising: at least one compound of the family of N-acylamino amides of formula (I); [Chem 10] at least one alkyl polyglycoside of formula (IV) below: R9(0)(G)x (iv) in which the radical R9 is a linear or branched C14-C24 alkyl radical, G represents a reduced sugar comprising from 5 to 6 carbon atoms; and x denotes a value ranging from 1 to 10 and preferably from 1 to 4. In particular, said composition also comprises at least one compound chosen from oxyethylenated sorbitan esters and fatty acid esters of a polyoxyalkylene glycol. Use for caring for, making up and/or cleansing keratin materials, in particular the skin.

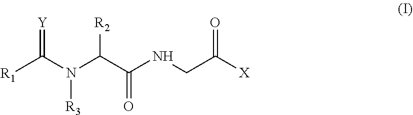

The invention relates to a composition, preferably a cosmetic composition, intended to be applied to keratin materials, in particular the skin and the lips, and the nails, comprising at least one compound of the N-acylamino amide family and at least one alkyl polyglycoside.

In particular, the invention relates to a cosmetic composition which is in the form of an oil-in-water (O/W) emulsion comprising at least one compound of the family of N-acylamino amides of formula (I) as defined below and at least one emulsifying surfactant of alkyl polyglycoside type.

The compounds of the N-acylamino amide family are compounds used for the cosmetic treatment of keratin materials, in particular the signs of skin ageing.

It is necessary for this compound to be formulated in a dissolved form, this compound being soluble in salt form, in order to fully exploit its activity, and it is also preferable for its dissolution to be maintained over time in order to avoid any recrystallization during storage of compositions comprising such a compound. The term “dissolved form” is intended to mean a dispersion of the derivatives according to the invention in a liquid, in the free molecular state, in particular in non-complexed form. No crystallization of the N-acylamino amide compounds is visible to the naked eye or under cross-polarized light microscopy.

In order to take advantage of these active agents, it is preferable to obtain the formulations that will make it possible to result in better bioavailability of this type of active agent in the skin after application of said formulations. For the purposes of the present application, the term “bioavailability” is intended to mean the molecular penetration of the active agent concerned into the live layers of the skin and in particular of the epidermis. It will be sought for the penetrated concentration to be as high as possible, so as to increase the amount of active agent reaching the live layers of the skin.

However, the improvement in the efficacy performance results of the active agents must not be obtained to the detriment of the cosmetic or sensory properties of the formulation comprising them.

It is thus sought to obtain cosmetic formulations comprising these active agents which have an increased efficacy in the cosmetic treatment of keratin materials, while at the same time having good cosmetic properties of the compositions.

An object of the present invention is specifically to provide a novel galenic formulation of the compounds of the N-acylamino amide family making it possible to improve the efficacy of a cosmetic composition to the benefit of the cosmetic, anti-ageing, treatment of keratin materials, it being possible for these compounds to be in particular present in a high content. This novel galenical formulation of the compounds of the N-acylamino amide family make it possible to improve the efficacy of the active agent for the treatment of keratin materials such as the skin, while at the same time having good cosmetic and/or sensory properties thereof.

The inventors have in fact discovered, unexpectedly, that the combination of at least one compound of the N-acylamino amide family with an alkyl polyglycoside, and in particular also with at least one compound chosen from oxyethylenated sorbitan esters and fatty acid esters of a polyoxyalkylene glycol, makes it possible to obtain a cosmetic emulsion having good stability, in particular after storage for 2 months at 4° C. or at 40° C. and/or 45° C., while at the same time exhibiting very good efficacy of the active agent, in particular exhibiting an improvement in the skin bioavailability obtained, and in particular an improvement in the skin bioavailability obtained with the compositions according to the invention compared to more conventional formulae comprising a compound of the N-acylamino amide family with an equal amount of active agent.

These compositions according to the invention, which are stable, make it possible to exhibit good efficacy of the active agent of the N-acylamino amide family, such as the compound [2-[acetyl-(3-trifluoromethylphenyl)amino]-3-methylbutyrylamino]acetic acid, the cosmetic treatment of keratin materials such as the skin, and in particular while at the same time having good cosmetic and/or sensory properties, for example soft on application, non-greasy and non-tacky during or after application.

More specifically, a subject of the invention is a composition, in particular a cosmetic composition, comprising, in a physiologically acceptable aqueous medium:

at least one compound of the family of N-acylamino amides of formula (I) as defined below; and

at least one alkyl polyglycoside of formula (IV) below:

R₉(O)(G)ₓ (IV)

in which the radical R₉ is a linear or branched C₁₄-C₂₄ alkyl radical;

G represents a reduced sugar comprising from 5 to 6 carbon atoms; and

x denotes a value ranging from 1 to 10 and preferably from 1 to 4.

In particular, a subject of the invention is advantageously a composition, in particular a cosmetic composition, in the form of an oil-in-water emulsion comprising:

at least one compound of the family of N-acylamino amides of formula (I) as defined below; and

at least one alkyl polyglycoside of formula (IV) below:

R₉(O)(G)ₓ (IV)

in which the radical R₉ is a linear or branched C₁₄-C₂₄ alkyl radical;

G represents a reduced sugar comprising from 5 to 6 carbon atoms; and

x denotes a value ranging from 1 to 10 and preferably from 1 to 4.

More particular, a subject of the invention is a composition, preferably a cosmetic composition, in the form of an oil-in-water emulsion comprising:

at least one compound of the family of N-acylamino amides of formula (I) as defined below; and

at least one alkyl polyglycoside of formula (IV) below:

R₉(O)(G)ₓ (IV)

in which the radical R₉ is a linear or branched C₁₄-C₂₄ alkyl radical;

G represents a reduced sugar comprising from 5 to 6 carbon atoms; and

x denotes a value ranging from 1 to 10, preferably 1 to 4; and

at least one compound chosen from oxyethylenated sorbitan esters and fatty acid esters of a polyoxyalkylene glycol, and mixtures thereof.

The composition obtained according to the invention, preferably the emulsion, has good stability over time, even at a temperature above ambient temperature (for example 40° C. or 45° C.). The term “stable composition” or “stable emulsion” is generally intended to mean a composition or emulsion which exhibits little or even no macroscopic and microscopic change (in colour, in odour, in viscosity, nor any variation in pH) after storage and/or temperature change, in particular after 2 months at 4° C., ambient temperature, 40° C. or 45° C.

A subject of the invention is also a cosmetic treatment process for caring for and/or making up keratin materials, which consists in applying to the keratin materials a composition as defined above.

A subject of the invention is also the use of said composition in the cosmetics or dermatology, preferably cosmetic, field, and in particular for caring for, protecting and/or making up keratin materials such as the skin, in particular bodily and/or facial skin, and in particular for preventing and/or treating the signs of ageing of keratin materials, such as the signs of skin ageing.

The composition according to the invention is intended for topical application and thus contains a physiologically acceptable medium. The term “physiologically acceptable medium” is intended to mean here a medium that is compatible with said keratin materials.

Within the context of the present invention, “keratin material” is intended to mean in particular the skin, the scalp, the nails, the mucous membranes such as the lips, and more particularly the skin (body, face, area around the eyes, eyelids).

In the text hereinbelow, the expression “at least one” is equivalent to “one or more” and, unless otherwise indicated, the limits of a range of values are included in that range.

### Compound of the N-acylamino amide Family

The compositions according to the invention comprise at least one compound of the N-acylamino amide family which corresponds to formula (I) below:

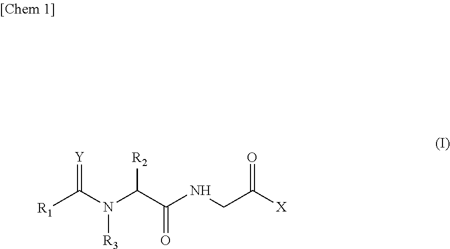

in which:

the radical Y represents O or S,

the radical R1 represents:

(i) a hydrogen atom;

(ii) a linear, branched or cyclic, saturated or unsaturated hydrocarbon-based radical comprising 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR; —P(O)—(OR)₂; —SO₂—OR;

with R and R′ representing, independently of one another, a linear, branched or cyclic, saturated or unsaturated hydrocarbon-based radical comprising 1 to 6 carbon atoms, which is optionally halogenated, or even perhalogenated,

it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; —Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated,

(iii) a radical chosen from the radicals —OR; —NH₂; —NHR; —NRR′; —NH—COR; —COOR; —COR;

with R and R′ representing, independently of one another, a linear, branched or cyclic, saturated or unsaturated hydrocarbon-based radical comprising 1 to 6 carbon atoms, which is optionally halogenated, or even perhalogenated,

it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated,

the radical R2 represents a linear, branched or cyclic, saturated or unsaturated hydrocarbon-based radical comprising 1 to 18 carbon atoms,

optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;

with R and R′ representing, independently of one another, a linear, branched or cyclic, saturated or unsaturated hydrocarbon-based radical comprising 1 to 6 carbon atoms, which is optionally halogenated, or even perhalogenated,

it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated,

the radical R3 represents a radical chosen from those of formula (II) or (III):

—A—C6H(5-y)-By (II)

—C6H(5-y′)-By′ (III)

in which:

y is an integer between 0 and 5 included, and y′ is an integer between 1 and 5 included;

A is a linear or branched, saturated or unsaturated divalent hydrocarbon-based radical comprising 1 to 18 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; —NO₂; —SO₂—OR;

with R and R′ representing, independently of one another, a linear, branched or cyclic, saturated or unsaturated hydrocarbon-based radical comprising 1 to 6 carbon atoms, which is optionally halogenated, or even perhalogenated,

it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated,

B is a linear or branched, saturated or unsaturated hydrocarbon-based radical comprising 1 to 18 carbon atoms,

optionally substituted with 1 to 5 groups, which may be identical or different, chosen from —OH; —OR;

O—COR; —SH; —SR; —S—COR; —NH₂; —NHR; —NRR′; —NH—COR; -Hal (halogen, or even perhalogen); —CN; —COOR; —COR; —NO₂; —SO₂—OR;

with R and R′ representing, independently of one another, a linear, branched or cyclic, saturated or unsaturated hydrocarbon-based radical comprising 1 to 6 carbon atoms, which is optionally halogenated, or even perhalogenated,

it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated;

the radical X represents a radical chosen from —OH, —OR4, —NH₂, —NHR4, —NR4R5, —SR4, —COOR4; —COR4;

with R4 and R5 representing, independently of one another, a linear, branched or cyclic, saturated or unsaturated hydrocarbon-based radical comprising 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen, or even perhalogen); —CN; —COOR; —COR; with R and R′ representing, independently of one another, a linear, branched or cyclic, saturated or unsaturated hydrocarbon-based radical comprising 1 to 6 carbon atoms, which is optionally halogenated, or even perhalogenated; it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated;

it being possible for said radicals R4 and R5 to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated, or even perhalogenated.

Also included in this definition are the inorganic or organic acid salts of said compounds, and also the optical isomers thereof, in isolated forms or as a racemic mixture.

The term “linear, branched or cyclic hydrocarbon-based radical” is intended to mean in particular the radicals of alkyl, aryl, aralkyl, alkylaryl, alkenyl and alkynyl type.

The C₆H₅ group present in the radical R3 must be included as an aromatic cyclic group.

Preferably, the radical Y represents oxygen.

Preferably, the radical R1 represents hydrogen or an optionally substituted linear or branched, saturated or unsaturated hydrocarbon-based radical comprising 1 to 12, and in particular 1, 2, 3, 4, 5 or 6 carbon atoms.

In particular, the substituents can be chosen from —OH, —OR and/or —P(O)—(OR)₂ with R representing a linear, branched or cyclic, saturated or unsaturated hydrocarbon-based radical comprising 1 to 6 carbon atoms, which is optionally halogenated, or even perhalogenated.

Preferentially, the radical R1 represents a methyl, ethyl, propyl or isopropyl radical, optionally substituted with an —OH or —P(O)—(OR)₂ group with R representing methyl, ethyl, propyl or isopropyl.

Preferably, the radical R2 represents an optionally substituted linear, branched or cyclic, saturated or unsaturated hydrocarbon-based radical comprising 1 to 12, in particular 1, 2, 3, 4, 5 or 6 carbon atoms.

In particular, the substituents can be chosen from —OH and —OR with R representing a linear, branched or cyclic, saturated or unsaturated hydrocarbon-based radical comprising 1 to 6 carbon atoms, which is optionally halogenated, or even perhalogenated.

Preferentially, the radical R2 represents a methyl, ethyl, propyl, isopropyl, n-butyl, ter-butyl or isobutyl radical.

Preferably, the radical R3 represents a radical of formula —C₆H(5-y′)-By′ for which y′=1, 2 or 3; or a radical of formula —A—C₆H(5-y)-By for which y=0, 1 or 2.

Preferably, A is an optionally substituted linear or branched, saturated or unsaturated, divalent hydrocarbon-based radical comprising 1 to 12 carbon atoms.

The substituents of A are preferably chosen from -Hal (halogen, or even perhalogen); —CN; —COOR; —NO₂; —SO₂-OR; with R representing a linear, branched or cyclic, saturated or unsaturated hydrocarbon-based radical comprising 1 to 6 carbon atoms, which is optionally halogenated, or even perhalogenated.

Preferably, B is an optionally substituted linear or branched, saturated or unsaturated hydrocarbon-based radical comprising 1 to 12 carbon atoms.

The substituents of B are preferably chosen from -Hal (halogen, or even perhalogen); —CN; —COOR; —NO₂; —SO₂-OR; with R representing a linear, branched or cyclic, saturated or unsaturated hydrocarbon-based radical comprising 1 to 6 carbon atoms, which is optionally halogenated, or even perhalogenated.

Preferentially, the radical R3 represents a group chosen from one of the formulae below:

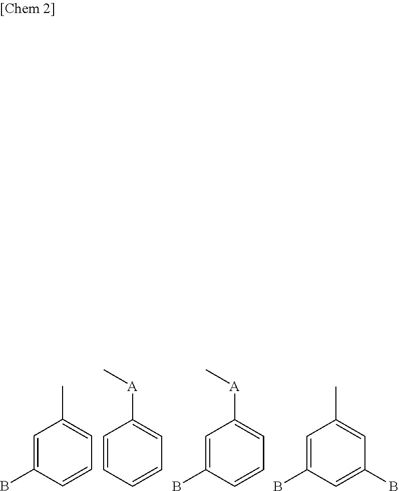

in which A and B have the meanings above.

In particular, the divalent radical A may be a methylene, an ethylene or a propylene.

The radical B is preferably a methyl, ethyl, propyl or isopropyl radical, substituted with one or more halogens, in particular chlorine, bromine, iodine or fluorine, and preferentially totally halogenated (perhalogenated), such as perfluorinated. Mention may in particular be made of the perfluoromethyl radical (—CF₃) as most particularly preferred.

Preferably, the radical X represents a radical chosen from —OH or —OR4 with R4 representing an optionally substituted linear, cyclic or branched, saturated or unsaturated hydrocarbon-based radical comprising 1 to 6 carbon atoms.

The substituents can be chosen from —OH and —OR with R representing a linear, branched or cyclic, saturated or unsaturated hydrocarbon-based radical comprising 1 to 6 carbon atoms, which is optionally halogenated, or even perhalogenated.

Preferentially, the radical X represents a radical chosen from —OH, —OCH₃, —OC₂H₅, —O—C₃H₇ or —OC₄H₉.

Among the compounds that are particularly preferred, mention may be made of:

[2-[acetyl-(3-trifluoromethylphenyl)amino]-3-methylbutyrylamino]acetic acid,

ethyl[2-[acetyl-(3-trifluoromethylphenyl)amino]-3-methylbutyrylamino]acetate,

[2-(acetylbenzylamino)-3-methylbutyrylamino]acetic acid,

ethyl[2-(acetylbenzylamino)-3-methylbutyrylamino]acetate,

ethyl(2-[benzyl[(diethoxyphosphoryl)acetyl]amino]-3-methylbutyrylamino)acetate.

The compounds according to the invention may be readily prepared by those skilled in the art on the basis of his general knowledge. A carboxylic acid, an aldehyde, an amino compound and an isonitrile can in particular be reacted together according to the Ugi reaction.

Of course, during the synthesis of compounds according to the invention, and depending on the nature of the different radicals present in the starting compounds, those skilled in the art can take care to protect some substituents so that they do not interact in the remainder of the reactions.

These compounds may be prepared according to the procedures described in application EP 1 292 608.

According to the invention, the compound of formula (I) that is particularly preferred is [2-[acetyl-(3-trifluoromethylphenyl)amino]-3-methylbutyrylamino]acetic acid.

The compound of the family of N-acylamino amides of formula (I), preferably [2-[acetyl-(3-trifluoromethylphenyl)amino]-3-methylbutyrylamino]acetic acid, may be present in the composition according to the invention in a content ranging from 0.001% to 50% by weight, relative to the total weight of the composition, more particularly in a content ranging from 0.005% to 15% by weight, and preferably ranging from 0.010% to 10% by weight.

Composition according to the invention

Preferentially, the composition, in particular cosmetic composition, according to the invention is in the form of an emulsion, in particular in the form of an oil-in-water emulsion, termed direct emulsion.

An oil-in-water emulsion is generally constituted of an aqueous liquid phase, in this case the continuous phase, and of an oily liquid phase, in this case the dispersed phase.

It is a dispersion of droplets of the oily phase in the aqueous phase.

Furthermore, an emulsion according to the present invention requires the presence of a surfactant or of an emulsifier to ensure its stability over time.

### Alkyl Polyglycosides

The composition according to the invention comprises at least one surfactant of alkyl polyglycoside type of formula (IV) below:

R₉(O)(G)ₓ (IV)

in which the radical R₉ is a linear or branched C₁₄-C₂₄ alkyl radical;

G represents a reduced sugar comprising from 5 to 6 carbon atoms; and

x denotes a value ranging from 1 to 10 and preferably from 1 to 4.

According to one particular embodiment of the invention, the alkyl polyglycoside(s) are chosen from the compounds of formula (IV′) below:

R₉′—O-G (IV′)

in which:

the radical R₉′ is a branched, preferably saturated, alkyl radical comprising from 14 to 24 carbon atoms, preferably from 18 to 22 carbon atoms, and G denotes a reduced sugar comprising from 5 to 6 carbon atoms, preferably a xylose residue.

For the purposes of the present invention, the term “alkyl polyglycoside” is intended to mean an alkylmonosaccharide (degree of polymerization 1) or an alkyl polysaccharide (degree of polymerization greater than 1). In the context of the present invention, the alkyl polyglycosides may be used alone or in the form of mixtures of several alkyl polyglycosides.

In a preferred embodiment, G may be chosen from glucose, dextrose, saccharose, fructose, galactose, maltose, maltotriose, lactose, cellobiose, mannose, ribose, dextran, talose, allose, xylose, levoglucan, cellulose or starch, and more preferentially denotes glucose, fructose, xylose or galactose.

It should also be noted that each unit of the polysaccharide part of the alkyl polyglycoside may be in α or β isomer form, in L or D form, and the configuration of the saccharide residue may be of furanoside or pyranoside type.

It is, of course, possible to use mixtures of alkyl polysaccharides, which may differ from one another in the nature of the borne alkyl unit and/or the nature of the bearing polysaccharide chain.

In one preferred embodiment of the invention, the alkyl polyglycoside is an alkyl polyglucoside, in which R₉ more particularly represents an oleyl radical (unsaturated C₁₈ radical) or isostearyl radical (saturated C₁₈ radical), G denotes glucose, x is a value ranging from 1 to 2, especially isostearyl glucoside or oleyl glucoside, and mixtures thereof.

In another preferred embodiment of the invention, the alkyl polyglycoside is an alkyl polyxyloside, in which R₉ more particularly represents an octyldodecyl radical, G denotes xylose and x is equal to 1, of formula:

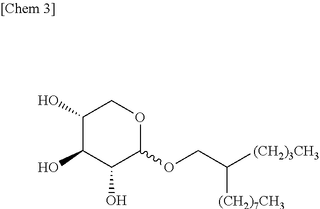

Advantageously, the surfactant of alkyl polyglycoside type is present in a content ranging from 0.01% to 50% by weight, particularly from 0.1% to 10% by weight and more particularly from 1% to 3% by weight relative to the total weight of the composition.

According to a particular mode of the invention, the alkyl polyglycoside may be used as a mixture with a coemulsifier, more especially with at least one fatty alcohol, and especially a fatty alcohol comprising from 14 to 24 carbon atoms and preferably a fatty alcohol bearing the same fatty chain as that of the alkyl polyglycoside.

In one particular embodiment of the invention, the mixture formed by the surfactant of alkyl polyglycoside type and the fatty alcohol is present in a content ranging from 0.01% to 50% by weight, more particularly from 0.1% to 10% by weight and more particularly of about 2% by weight relative to the total weight of the composition.

The alkyl polyglycoside(s), preferably of octyldodecyl xyloside, is (are) present in a content of active material (AM) ranging from 0.005% to 15% by weight, in particular from 0.01% to 5% by weight, preferably of in particular from 0.1% to 2% by weight relative to the total weight of the composition according to the invention.

In one particular embodiment, when the alkyl polyglycoside is isostearyl glucoside, it is advantageously mixed with isostearyl alcohol. Such a mixture is sold especially under the name Montanov WO18 by the company SEPPIC.

In another embodiment, when the alkyl polyglycoside is oleyl glucoside, it is advantageously mixed with oleyl alcohol, optionally in the form of a self-emulsifying composition, as described, for example, in WO 92/06778.

In another embodiment of the invention, when the alkyl polyglycoside is octyldodecyl xyloside, it is advantageously mixed with octyldodecanol.

Thus, in one particularly preferred embodiment according to the invention, the alkyl polyglycoside is octyldodecyl xyloside and the fatty alcohol is octyldodecanol.

Such a mixture is sold especially under the name Fluidanov 20X® by the company SEPPIC (constituted of about 20% to 30% of octyldodecyl xyloside and of about 70% to 80% of octyldodecanol).

In such an embodiment of the invention, the amount of octyldodecyl xyloside used in the composition according to the invention ranges from 20% to 30% by weight relative to the total weight of the mixture of octyldodecyl xyloside and of octyldodecanol.

Similarly, the amount of octyldodecanol used in the composition according to the invention ranges from 70% to 80% by weight relative to the total weight of the mixture of octyldodecyl xyloside and of octyldodecanol.

Advantageously, the mixture of octyldodecyl xyloside and of octyldodecanol is present in a content ranging from 0.02% to 10% by weight, particularly from 1% to 5% by weight and more particularly of about 2% by weight relative to the total weight of the composition.

According to a particular embodiment, the alkyl polyglycoside may constitute the main surfactant system of the composition.

The term “main surfactant system” is intended to mean a system which, in its absence, does not lead to the formation of a stable composition.

The term “main” is intended to mean that any additional surfactant, other than the alkyl polyglycoside(s) according to the invention, or the mixture of the alkyl polyglycoside according to the invention with a coemulsifier, more especially with at least one fatty alcohol, and especially a fatty alcohol bearing the same fatty chain as that of the alkyl polyglycoside, is present in a content not exceeding 1% and preferably not exceeding 0.5%.

### Additional Emulsifying Surfactant

In one preferred embodiment of the invention, the composition according to the invention also advantageously comprises at least one additional emulsifying surfactant chosen from oxyethylenated sorbitan esters and fatty acid esters of a polyoxyalkylene glycol.

### Oxyethylenated Sorbitan Esters

The oxyethylenated sorbitan esters that can be used according to the invention comprise in particular the oxyethylenated compounds of C₈-C₃₀ fatty acid monoesters and polyesters of sorbitan, having from 1 to 50 ethylene oxide units. Use is preferably made of the oxyethylenated compounds of C₁₀-C₂₄ fatty acid monoesters and polyesters of sorbitan, having from 4 to 30 and preferably from 15 to 25 ethylene oxide units.

The C₁₀-C₂₄ fatty acids used are preferably lauric acid, palmitic acid, stearic acid and oleic acid.

Such compounds also known under the name of polysorbates. They are, inter alia, sold under the name Tween by the company Croda. Mention may be made for example of: oxyethylene sorbitan monolaurate with 4 OE, sold under the name Tween 21, oxyethylene sorbitan monolaurate with 20 OE, sold under the name Tween 20, oxyethylene sorbitan monopalmitate with 20 OE, sold under the name Tween 40, oxyethylene sorbitan monostearate with 20 OE, sold under the name Tween 60, oxyethylene sorbitan tristearate with 20 OE, sold under the name Tween 65, oxyethylene sorbitan monooleate with 20 OE, sold under the name Tween 80, oxyethylene sorbitan monooleate with 5 OE, sold under the name Tween 81, oxyethylene sorbitan trioleate with 20 OE, sold under the name Tween 85.

Oxyethylene sorbitan monolaurate with 4 OE is also sold under the name Tego SML 21 by the company Evonik Goldschmidt.

In the present document, and in a manner well known per se, a “compound with X OE” denotes an oxyethylenated compound comprising X oxyethylene units per molecule.

Thus, the fatty acid of the oxyethylenated sorbitan ester may be a saturated or unsaturated fatty acid.

The preferred sorbitan esters are oxyethylene sorbitan monostearate with 20 OE, oxyethylene sorbitan monooleate with 20 OE, oxyethylene sorbitan monolaurate with 20 OE, and mixtures thereof.

According to an even more preferred embodiment, the composition according to the invention comprises oxyethylene sorbitan monolaurate with 20 OE.

Polysorbate 20 oxyethylene sorbitan monolaurate with 20 OE is available in particular under the trade name Tween 20-LQ-(AP) from Croda or Tego SML 20 from Evonik Goldschmidt.

The oxyethylenated sorbitan ester(s) is (are) present in the compositions according to the invention in a content ranging from 0.05% to 5%, preferably from 0.1% 3% and preferentially from 0.15% to 1.0% by weight relative to the total weight of said composition.

### Fatty Acid Esters of a Polyoxyalkylene Glycol

The fatty acid ester of polyoxyalkylene glycol present in the composition according to the invention has water-in-oil emulsifying properties.

Preferably, the fatty acid ester of a polyoxyalkylene glycol is a polyhydroxylated fatty acid ester of polyethylene glycol.

The fatty acid ester of a polyoxyalkylene glycol may be a monoester or a polyester, in particular a diester or a triester.

The fatty acid comprises from 12 to 22 carbon atoms, preferably from 12 to 20 carbon atoms, preferentially from 14 to 18 carbon atoms. It may be chosen in particular from oleic acid, palmitic acid and stearic acid, and mixtures thereof.

The polyethylene glycol may comprise from 8 to 120 mol of ethylene oxide, preferably from 4 to 50 mol of ethylene oxide and more preferably from 20 to 40 mol of ethylene oxide.

According to one embodiment, use is made of an ester, preferably a diester, of polyethylene glycol comprising from 20 to 40 mol of ethylene oxide and of a polyhydroxylated fatty acid, said polyhydroxylated fatty acid comprising from 14 to 18 carbon atoms, in particular polyhydroxystearic acid.

In particular, the fatty acid ester of a polyoxyalkylene glycol is in the form of a block polymer, preferably of ABA structure, comprising poly(hydroxylated ester) blocks and polyethylene glycol blocks.

The fatty acid ester of said polymer bears a chain comprising from 12 to 20 carbon atoms and preferably from 14 to 18 carbon atoms.

The polyethylene glycol blocks of said emulsifying polymer as defined above preferably comprise from 4 to 50 mol of ethylene oxide and more preferably from 20 to 40 mol of ethylene oxide.

Preferably, the additional emulsifying surfactant used is a polyethylene glycol polyhydroxystearate, preferably polyethylene glycol dipolyhydroxystearate with 30 OE (INCI name PEG-30 dipolyhydroxystearate) which is available under the trade name Arlacel P135 from the company Croda, or under the trade name Cithrol DPHS-SO-(MV) from the company Croda.

The fatty acid ester(s) of a polyoxyalkylene glycol is (are) preferably present in the compositions according to the invention in a content ranging from 0.05% to 5% by weight, relative to the total weight of the composition, and in particular from 0.1% to 3% by weight and preferentially from 0.15% to 1% relative to the total weight of the composition.

In one particular embodiment, in the composition according to the invention, the weight ratio of said alkyl polyglycoside(s), in particular octyldodecyl xyloside, with a fatty alcohol/the additional emulsifying surfactant as defined above is from 0.5 to 1.5, preferably is 1.0.

### Aqueous Phase

In the context of the present invention, the aqueous phase comprises water and optionally at least one hydrophilic adjuvant. The term “adjuvant” is intended to mean an active agent or additive, known in the prior art to have a physical, chemical and/or biological property of interest in the context of skincare, dermatology, cosmetics and/or makeup.

According to one aspect of the invention, the aqueous phase may represent approximately 10% to 95% by weight, preferably approximately 20% to 90% by weight and preferably approximately 40% to 85% by weight, relative to the total weight of the composition.

In one particular embodiment, the water preferably represents at least approximately 30% of the weight, preferably at least approximately 40% by weight, preferably at least approximately 50% by weight, relative to the total weight of the composition. In particular, the water may represent, for example, approximately 30% to 95% by weight, preferably approximately 40% to 90% by weight and preferably approximately 50% to 80% by weight, relative to the total weight of the composition.

Hydrophilic adjuvants that may in particular be mentioned, in a non-limiting way, include monoalcohols containing 2 to 8 carbon atoms, for instance ethanol and isopropanol, and polyols, for instance glycerol, glycols, for instance pentylene glycol, propylene glycol, butylene glycol, dipropylene glycol, isoprene glycol and polyethylene glycols such as PEG-8; sorbitol; sugars such as glucose, fructose, maltose, lactose or sucrose; and mixtures thereof.

According to one variant, the aqueous phase is gelled.

The composition according to the invention may comprise at least one hydrophilic gelling agent.

For the purposes of the present invention, the term “hydrophilic gelling agent” is intended to mean a compound that is capable of gelling the aqueous phase of the compositions according to the invention.

The hydrophilic gelling agent is thus present in the aqueous phase of the composition.

The gelling agent may be water-soluble or water-dispersible.

The hydrophilic gelling agent may be chosen from polymeric gelling agents that are natural or of natural origin and synthetic polymeric gelling agents, and mixtures thereof.

### Polymeric Gelling Agents that are Natural or of Natural Origin

The polymeric hydrophilic gelling agents that are suitable for use in the invention may be natural or of natural origin.

For the purposes of the invention, the term “of natural origin” is intended to denote polymeric gelling agents obtained by modification of natural polymeric gelling agents.

These gelling agents may be particulate or non-particulate.

More specifically, these gelling agents fall within the category of polysaccharides.

In general, polysaccharides may be divided into several categories.

Thus, the polysaccharides that are suitable for use in the invention may be homopolysaccharides such as fructans, glucans, galactans and mannans or heteropolysaccharides such as hemicellulose.

Similarly, they may be linear polysaccharides such as pullulan or branched polysaccharides such as gum arabic and amylopectin, or mixed polysaccharides such as starch.

More particularly, the polysaccharides that are suitable for use in the invention may be distinguished according to whether or not they are starchy.

Examples of non-starchy polysaccharides that may be mentioned include carrageenans, in particular kappa carrageenan, gellan gum, agar-agar, xanthan gum, alginate-based compounds, in particular sodium alginate, scleroglucan gum, guar gum, inulin and pullulan, and mixtures thereof.

Mention may also be made of hyaluronic acid, or a salt thereof such as the sodium salt, such as sodium hyaluronate.

### Synthetic Polymeric Gelling Agents

For the purposes of the invention, the term “synthetic” means that the polymer is neither naturally existing nor a derivative of a polymer of natural origin.

The synthetic polymeric hydrophilic gelling agent under consideration according to the invention may or may not be particulate.

For the purposes of the invention, the term “particulate” means that the polymer is in the form of particles, preferably spherical particles.

As emerges from the text hereinbelow, the polymeric hydrophilic gelling agent is advantageously chosen from crosslinked acrylic homopolymers or copolymers; associative polymers, in particular associative polymers of polyurethane type; polyacrylamides and crosslinked and/or neutralized 2-acrylamido-2-methylpropanesulfonic acid polymers and copolymers; modified or unmodified carboxyvinyl polymers, and mixtures thereof, especially as defined below.

### Particulate Synthetic Polymeric Gelling Agents

They are preferably chosen from crosslinked polymers.

They may notably be crosslinked acrylic homopolymers or copolymers, which are preferably partially neutralized or neutralized, and which are in particulate form.

According to one embodiment, the particulate gelling agent according to the present invention is chosen from crosslinked sodium polyacrylates. Preferably, it has in the dry or non-hydrated state a mean size of less than or equal to 100 μm and preferably less than or equal to 50 μm. The mean size of the particles corresponds to the mass-average diameter (D50) measured by laser particle size analysis or another equivalent method known to those skilled in the art.

Thus, preferably, the particulate gelling agent according to the present invention is chosen from crosslinked sodium polyacrylates, preferably in the form of particles with a mean size (or mean diameter) of less than or equal to 100 microns, more preferably in the form of spherical particles.

As examples of crosslinked sodium polyacrylates, mention may be made of those sold under the names Octacare X100, X110 and RM100 by the company Avecia, those sold under the names Flocare GB300 and Flosorb 500 by the company SNF, those sold under the names Luquasorb 1003, Luquasorb 1010, Luquasorb 1280 and Luquasorb 1110 by the company BASF, those sold under the names Water Lock G400 and G430 (INCI name: Acrylamide/Sodium acrylate copolymer) by the company Grain Processing.

Mention may also be made of crosslinked polyacrylate microspheres, for instance those sold under the name Aquakeep® 10 SH NF by the company Sumitomo Seika.

### Non-Particulate Synthetic Polymeric Gelling Agents

This family of gelling agents may be detailed under the following subfamilies:
1. Associative polymers,
2. Polyacrylamides and crosslinked and/or neutralized 2-acrylamido-2-methylpropanesulfonic acid polymers and copolymers, and
3. Modified or unmodified carboxyvinyl polymers.

More particularly, the polymers used that are suitable as aqueous gelling agent for the invention may be crosslinked or non-crosslinked homopolymers or copolymers comprising at least the 2-acrylamidomethylpropanesulfonic acid (AMPS®) monomer, in a form partially or totally neutralized with a mineral base other than aqueous ammonia, such as sodium hydroxide or potassium hydroxide.

They are preferably totally or almost totally neutralized, i.e. at least 90% neutralized.

These AMPS® polymers according to the invention may be crosslinked or non-crosslinked.

When the polymers are crosslinked, the crosslinking agents may be chosen from the polyolefinically unsaturated compounds commonly used for crosslinking polymers obtained by radical polymerization.

The water-soluble or water-dispersible AMPS® polymers of the invention preferably have a molar mass ranging from 50 000 g/mol to 10 000 000 g/mol, preferably from 80 000 g/mol to 8 000 000 g/mol, and even more preferably from 100 000 g/mol to 7 000 000 g/mol.

As water-soluble or water-dispersible AMPS homopolymers suitable for use in the invention, mention may be made, for example, of crosslinked or non-crosslinked polymers of sodium acrylamido-2-methylpropanesulfonate, such as that used in the commercial product Simulgel 800 (CTFA name: Sodium Polyacryloyldimethyl Taurate), crosslinked ammonium acrylamido-2-methylpropanesulfonate polymers (INCI name: Ammonium polydimethyltauramide) such as those described in patent EP 0 815 928 B1 and such as the product sold under the trade name Hostacerin AMPS® by the company Clariant.

As water-soluble or water-dispersible AMPS copolymers in accordance with the invention, examples that may be mentioned include:

crosslinked acrylamide/sodium acrylamido-2-methylpropanesulfonate copolymers, such as that used in the commercial product Sepigel 305 (CTFA name: Polyacrylamide/C₁₃-C₁₄ isoparaffin/laureth-7) or that used in the commercial product sold under the name Simulgel 600 (CTFA name: Acrylamide/sodium acryloyldimethyltaurate/isohexadecane/polysorbate-80) by the company SEPPIC;

copolymers of AMPS® and of vinylpyrrolidone or vinylformamide, such as that used in the commercial product sold under the name Aristoflex AVC® by the company Clariant (CTFA name: Ammonium Acryloyldimethyltaurate/VP copolymer) but neutralized with sodium hydroxide or potassium hydroxide;

copolymers of AMPS® and of sodium acrylate, for instance the AMPS/sodium acrylate copolymer, such as that used in the commercial product sold under the name Simulgel EG® by the company SEPPIC or under the trade name Sepinov EM (CTFA name: Hydroxyethyl acrylate/Sodium acryloyldimethyltaurate copolymer);

copolymers of AMPS® and of hydroxyethyl acrylate, for instance the AMPS®/hydroxyethyl acrylate copolymer, such as that used in the commercial product sold under the name Simulgel NS® by the company SEPPIC (CTFA name: Hydroxyethyl acrylate/Sodium acryloyldimethyltaurate copolymer (and) squalane (and) polysorbate 60), or such as the product sold under the name sodium acrylamido-2-methylpropanesulfonate/hydroxyethyl acrylate copolymer, such as the commercial product Sepinov EMT 10 (INCI name: Hydroxyethyl acrylate/Sodium acryloyldimethyltaurate copolymer).

The modified or unmodified carboxyvinyl polymers may be copolymers derived from the polymerization of at least one monomer (a) chosen from α,β-ethylenically unsaturated carboxylic acids or esters thereof, with at least one ethylenically unsaturated monomer (b) comprising a hydrophobic group.

Among said abovementioned polymers, the ones that are most particularly preferred according to the present invention are acrylate/C₁₀-C₃₀-alkyl acrylate copolymers (INCI name: Acrylates/C₁₀₋₃₀ Alkyl acrylate Crosspolymer) such as the products sold by the company Lubrizol under the trade names Pemulen TR-1, Pemulen TR-2, Carbopol 1382, Carbopol EDT 2020 and Carbopol Ultrez 20 Polymer, and even more preferentially Pemulen TR-2.

Among the modified or unmodified carboxyvinyl polymers, mention may also be made of sodium polyacrylates such as those sold under the name Cosmedia SP® containing 90% solids and 10% water, or Cosmedia SPL® as an inverse emulsion containing about 60% solids, an oil (hydrogenated polydecene) and a surfactant (PPG-5 Laureth-5), both sold by the company Cognis.

Mention may also be made of partially neutralized sodium polyacrylates that are in the form of an inverse emulsion comprising at least one polar oil, for example the product sold under the name Luvigel® EM by the company BASF.

The modified or unmodified carboxyvinyl polymers may also be chosen from crosslinked (meth)acrylic acid homopolymers.

For the purposes of the present application, the term “(meth)acrylic” intended to mean “acrylic or methacrylic”.

Examples that may be mentioned include the products sold by Lubrizol under the names Carbopol 910, 934, 940, 941, 934 P, 980, 981, 2984, 5984 and Carbopol Ultrez 10 Polymer, or by 3V-Sigma under the name Synthalen® K, Synthalen® L or Synthalen® M.

Among the modified or unmodified carboxyvinyl polymers, mention may be made in particular of Carbopol (CTFA name: carbomer) and Pemulen (CTFA name: Acrylates/C₁₀₋₃₀ alkyl acrylate crosspolymer) sold by the company Lubrizol.

In one particular embodiment, the composition according to the invention may comprise an anionic terpolymer.

The anionic terpolymer used according to the invention is a linear or branched and/or crosslinked terpolymer, of at least one monomer (1) bearing an acid function in free form, which is partially or totally salified with a non-ionic monomer (2) chosen from N,N-dimethylacrylamide and 2-hydroxyethyl acrylate and (3) at least one polyoxyethylenated alkyl acrylate monomer (3) of formula (V) below:

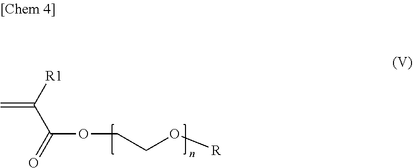

in which R1 represents a hydrogen atom, R represents a linear or branched C₂-C₈ alkyl radical and n represents a number ranging from 1 to 10.

The acid function of the monomer (1) is notably a sulfonic acid or phosphonic acid function, said functions being in free or partially or totally salified form.

The monomer (1) may be chosen from styrenesulfonic acid, ethylsulfonic acid and 2-methyl-2-[(1-oxo-2-propenyl)amino]-1-propanesulfonic acid (also known as acryloyldimethyl taurate), in free or partially or totally salified form. It is present in the anionic terpolymer of the invention preferably in molar proportions of between 5 mol % and 95 mol % and more particularly between 10 mol % and 90 mol %.

The monomer (1) will more particularly be 2-methyl-2-[(1-oxo-2-propenyl)amino]-1-propanesulfonic acid in free or partially or totally salified form.

The acid function in partially or totally salified form will preferably be an alkali metal salt such as a sodium or potassium salt, an ammonium salt, an amino alcohol salt such as a monoethanolamine salt, or an amino acid salt such as a lysine salt.

The monomer (2) is preferably present in the anionic terpolymer of the invention in molar proportions of between 4.9 mol % and 90 mol %, more particularly between 9.5 mol % and 85 mol % and even more particularly between 19.5 mol % and 75 mol %.

In formula (V), examples of linear C₈-C₁₆ alkyl radicals that may be mentioned include octyl, decyl, undecyl, tridecyl, tetradecyl, pentadecyl and hexadecyl.

In formula (I), examples of branched C₈-C₁₆ alkyl radicals that may be mentioned include 2-ethylhexyl, 2-propylheptyl, 2-butyloctyl, 2-pentylnonyl, 2-hexyldecyl, 4-methylpentyl, 5-methylhexyl, 6-methylheptyl, 15-methylpentadecyl, 16-methylheptadecyl and 2-hexyloctyl.

According to one particular form of the invention, in formula (V), R denotes a C₁₂-C₁₆ alkyl radical.

According to one particular form of the invention, in formula (V), n ranges from 3 to 5.

Tetraethoxylated lauryl acrylate will more particularly be used as monomer of formula (V).

The monomer (3) of formula (V) is preferably present in the anionic terpolymer of the invention in molar proportions of between 0.1 mol % and 10 mol % and more particularly between 0.5 mol % and 5 mol %.

According to a particular mode of the invention, the anionic terpolymer is crosslinked and/or branched with a diethylenic or polyethylenic compound in the proportion expressed relative to the total amount of monomers used, from 0.005 mol % to 1 mol %, preferably from 0.01 mol % to 0.5 mol % and more particularly from 0.01 mol % to 0.25 mol %.

The crosslinking agent and/or the branching agent is preferably chosen from

ethylene glycol dimethacrylate, diallyloxyacetic acid or a salt thereof, such as sodium diallyloxyacetate, tetraallyloxyethane, ethylene glycol diacrylate, diallylurea, triallylamine, trimethylolpropane triacrylate and methylenebis(acrylamide), or mixtures thereof.

The anionic terpolymer may contain additives such as complexing agents, transfer agents or chain-limiting agents.

Use will be made more particularly of an anionic terpolymer of 2-methyl-2-[(1-oxo-2-propenyl)amino]-1-propanesulfonic acid partially or totally salified in the form of ammonium salt, N,N-dimethylacrylamide and tetraethoxylated lauryl acrylate, crosslinked with trimethylolpropane triacrylate, under the INCI name: Polyacrylate Crosspolymer-6, such as the product sold under the trade name Sepimax Zen® by the company SEPPIC.

The hydrophilic gelling agent(s) may be used in a proportion of from 0.1% to 7% by weight and in particular from 0.5% to 4% by weight relative to the total weight of the composition.

The composition according to the invention may also comprise at least one additional compound chosen from fillers, nacres, pigments or the additional active agents other than the compounds of formula (I) according to the invention, such as moisturizing or anti-ageing active agents, and sunscreens.

Said additional compound is advantageously present in a content ranging from 0.01% to 15% by weight and particularly from 0.1% to 8% by weight relative to the total weight of the composition.

### Oily Phase

The oily phase is constituted of at least one oil or of any other fatty substance and lipophilic constituent which may be present in the composition of the invention, including the emulsifier. Any cosmetically acceptable oil may be used.

The term “oil” is intended to mean a fatty substance which is liquid at ambient temperature (25° C.).

According to one aspect of the invention, the oily phase may represent approximately 1% to 50% by weight, preferably approximately 3% to 40% by weight, and preferably represents approximately 5% to 30% by weight relative to the total weight of the composition.

As oils that may be used in the composition of the invention, examples that may be mentioned include:

hydrocarbon-based oils of animal origin, such as perhydrosqualene and squalane;

hydrocarbon-based oils of plant origin, such as liquid triglycerides of C₄-C₁₀ fatty acids, for instance heptanoic or octanoic acid triglycerides, or alternatively, for example, sunflower oil, corn oil, soya bean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those available under the trade names Miglyol 810, Miglyol 812 and Miglyol 818 by the company Dynamit Nobel, jojoba oil and rhea butter oil;

synthetic esters and ethers, especially of fatty acids, for instance the oils of formulae R1COOR2 and R1OR2 in which R1 represents the residue of a C₈ to C₂₉ fatty acid and R2 represents a C₃ to C₃₀ branched or unbranched hydrocarbon-based chain, for instance Purcellin oil, isononyl isononanoate, isopropyl myristate, 2-ethylhexyl palmitate, 2-octyldodecyl stearate, 2-octyldodecyl erucate or isostearyl isostearate; hydroxylated esters, for instance isostearyl lactate, octyl hydroxystearate, octyldodecyl hydroxystearate, diisostearyl malate or triisocetyl citrate; fatty alcohol heptanoates, octanoates or decanoates; polyol esters, for instance propylene glycol dioctanoate, neopentyl glycol diheptanoate and diethylene glycol diisononanoate; and pentaerythritol esters, for instance pentaerythrityl tetraisostearate (Prisorine 3631);

linear or branched hydrocarbons of mineral or synthetic origin, such as volatile or non-volatile liquid paraffins, and derivatives thereof, petroleum jelly, polydecenes, and hydrogenated polyisobutene such as Parleam oil;

silicone oils, for instance volatile or non-volatile polymethylsiloxanes (PDMSs) containing a linear or cyclic silicone chain, which are liquid or pasty at ambient temperature, in particular cyclopolydimethylsiloxanes (cyclomethicones) such as cyclohexasiloxane and cyclopentasiloxane; polydimethylsiloxanes (or dimethicones) comprising alkyl, alkoxy or phenyl groups, which are pendant or at the end of a silicone chain, these groups containing from 2 to 24 carbon atoms; phenylsilicones, for instance phenyl trimethicones, phenyl dimethicones, phenyltrimethylsiloxydiphenylsiloxanes, diphenyl dimethicones, diphenylmethyldiphenyltrisiloxanes, 2-phenylethyl trimethylsiloxysilicates and polymethylphenylsiloxanes;

fatty alcohols containing from 8 to 26 carbon atoms, for instance cetyl alcohol, stearyl alcohol and a mixture thereof (cetylstearyl alcohol);

partially hydrocarbon-based and/or silicone-based fluoro oils, such as those described in JP-A-2-295 912;

and mixtures thereof.

The term “hydrocarbon-based oil” hereinabove is intended to mean any oil mainly comprising carbon and hydrogen atoms, and possibly ester, ether, fluoro, carboxylic acid and/or alcohol groups.

In addition to the oils indicated above, the oily phase according to the invention may contain other fatty substances in the oily phase, such as C₈ to C₃₀ fatty acids, for instance stearic acid; silicone resins such as trifluoromethyl(C₁-C₄ alkyl) dimethicone and trifluoropropyl dimethicone; silicone gums (dimethiconol), non-emulsifying silicone elastomers, for instance the products sold under the names KSG 6 and KSG 16 by the company Shin-Etsu, under the names Trefil, BY29 and EPSX by the company Dow Corning, or under the name Gransil by the company Grant Industries; waxes, for example mineral waxes, waxes of animal origin, for instance beeswax, waxes of plant origin, hydrogenated oils that are solid at 25° C., fatty esters and glycerides that are solid at 25° C., synthetic waxes and silicone waxes; and mixtures thereof.

Finally, the oily phase may contain one or more waxes chosen from those described above, and in particular a synthetic wax such as polymethylene wax or polyethylene wax, or alternatively one or more pasty fatty substances such as petroleum jelly.

According to one variant of the invention, the oily phase is gelled.

The composition according to the invention may thus comprise at least one lipophilic gelling agent or thickener.

For the purposes of the present invention, the term “lipophilic gelling agent” is intended to mean a compound that is capable of gelling the oily phase of the compositions according to the invention.

The lipophilic gelling agent is thus present in the oily phase of the composition. The gelling agent is liposoluble or lipodispersible.

As emerges from the text hereinbelow, the lipophilic gelling agent is advantageously chosen from particulate gelling agents, silicas, dextrin esters and polymers containing hydrogen bonding, polyamides, and mixtures thereof.

Thus, according to one particular embodiment, the oily phase may also comprise one or more lipophilic adjuvant(s), such as, for example, one or more oily thickeners.

By way of thickener, mention may be made, for example, of organomodified clays, which are clays treated with compounds chosen in particular from quaternary amines and tertiary amines. Organomodified clays that may be mentioned include organomodified bentonites such as those which are commercially available under the name Bentone from the company Rheox, for instance those modified with distearyldimethylammonium chloride (Bentone 38 and Bentone 34), or the product modified with stearylbenzyldimethylammonium chloride (Bentone 27).

The oily-phase thickener may advantageously be chosen from fatty acid esters of glycerol, and in particular glycerol triesters such as glyceryl tristearate (tristearine), such as the mixture of acetylated glycol stearate and of glyceryl tristearate, sold under the name Unitwix by the company United Guardian.

The oily-phase thickener may also be chosen from fatty acid esters of dextrin, such as, especially, dextrin palmitate, especially those sold under the name Rheopearl by the company Chiba Flour Milling.

The thickener(s) may be present in an amount ranging, for example, from approximately 0.1% to 5% by weight, preferably approximately 0.1% to 3% by weight and preferably approximately 0.2% to 2% by weight, relative to the total weight of the composition.

For the purposes of the invention, the term “volatile oil” is intended to mean an oil that is capable of evaporating on contact with the skin or the keratin fibre in less than one hour, at ambient temperature and atmospheric pressure.

The volatile oils of the invention are volatile cosmetic oils, which are liquid at ambient temperature, having a nonzero vapor pressure, at ambient temperature and atmospheric pressure, ranging in particular from 0.13 Pa to 40 000 Pa (10-3 to 300 mmHg), in particular ranging from 1.3 Pa to 13 000 Pa (0.01 to 100 mmHg) and more particularly ranging from 1.3 Pa to 1300 Pa (0.01 to 10 mmHg).

The term “non-volatile oil” is intended to mean an oil that remains on the skin or the keratin fibre at ambient temperature and atmospheric pressure for at least several hours, and that especially has a vapour pressure strictly less than 10⁻³ mmHg (0.13 Pa).

According to one preferred embodiment, the composition according to the invention comprises a volatile oil which is a volatile silicone oil, more particularly is a volatile cyclic silicone oil.

As volatile silicone oils that may be used in the invention, mention may be made of linear or cyclic silicones with a viscosity at ambient temperature of less than 8 centistokes (cSt) (8×10⁻⁶m²/s), and in particular containing from 2 to 10 silicon atoms and in particular from 2 to 7 silicon atoms, these silicones optionally comprising alkyl or alkoxy groups containing from 1 to 10 carbon atoms.

As volatile silicone oils that may be used in the invention, mention may notably be made of dimethicones with viscosities of 5 and 6 cSt, octamethylcyclotetrasiloxane, decamethylcyclopentasiloxane or cyclopentasiloxane, dodecamethylcyclohexasiloxane or cyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Mention may also be made of the volatile linear alkyltrisiloxane oils of general formula (VI):

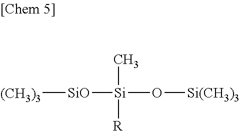

in which R represents an alkyl group containing from 2 to 4 carbon atoms and of which one or more hydrogen atoms may be substituted with one or more fluorine or chlorine atoms.

Among the oils of general formula (I), mention may be made of:

3-butyl-1,1,1,3,5,5,5-heptamethyltrisiloxane,

3-propyl-1,1,1,3,5,5,5-heptamethyltrisiloxane, and

3-ethyl-1,1,1,3,5,5,5-heptamethyltrisiloxane,

corresponding to the oils of formula (I) for which R is, respectively, a butyl group, a propyl group or an ethyl group.

The proportion of volatile oil(s) relative to the total amount of oils preferably ranges from 50% to 100% by weight.

Preferably, the composition according to the invention comprises at least one volatile oil chosen from cyclopentasiloxane, cyclohexasiloxane, dodecamethylpentasiloxane and a mixture thereof.

Advantageously, the oily phase of the composition according to the invention comprises cyclopentasiloxane and/or cyclohexasiloxane.

Cyclohexasiloxane, or dodecamethylcyclohexasiloxane, is in particular available under the trade name Xiameter PMX-0246 Cyclohexasiloxane® from the company Dow Corning.

Cyclopentasiloxane, or decamethylcyclopentasiloxane, is in particular available under the trade name Xiameter PMX-0245 Cyclopentasiloxane® from the company Dow Corning.

According to one aspect of the invention, the volatile oil(s) is (are) present in a content ranging from 0% to 50% by weight, more particularly in a content ranging from 1% to 30% by weight, and preferably ranging from 5% to 20% by weight, relative to the total weight of the composition.

In one preferred embodiment, the composition according to the invention also comprises at least one C₆-C₂₂ N-acylamino acid ester.

These derivatives are especially those described in patent application EP 1 269 986.

The N-acylamino acid ester(s) that can be used in according to the invention are of formula VII:

R′1(CO)N(R′2)CH(R′3)(CH2)n(CO)OR′4 (VII)

in which:

n is an integer equal to 0, 1 or 2,

R′₁ represents a linear or branched C₅-C₂₁ alkyl or alkenyl radical,

R′₂ represents a hydrogen atom or a C₁ to C₃ alkyl group,

R′₃ represents a radical chosen from the group formed by a hydrogen atom, a methyl group, an ethyl group and a linear or branched C₃ or C₄ alkyl radical,

R′₄ represents a linear or branched C₁ to C₁₀ alkyl or C₂ to C₁₀ alkenyl radical, or a sterol residue.

Preferably, the group R′₁(CO)— is an acyl group of an acid chosen from the group formed by capric acid, lauric acid, myristic acid, palmitic acid, stearic acid, behenic acid, linoleic acid, linolenic acid, oleic acid, isostearic acid, 2-ethylhexanoic acid, coconut oil fatty acids and palm kernel oil fatty acids. These fatty acids may also contain a hydroxyl group. Even more preferably, it will be lauric acid.

The —N(R′₂)CH(R′₃)(CH₂)ₙ(CO)— part of the amino acid ester is preferably chosen from the following amino acids: glycine, alanine, valine, leucine, isoleucine, serine, threonine, proline, hydroxyproline, β-alanine, aminobutyric acid, aminocaproic acid, sarcosine, or N-methyl-β-alanine.

Even more preferably, it will be sarcosine.

The part of the amino acid esters corresponding to the group OR′₄ may be obtained from alcohols chosen from the group formed by methanol, ethanol, propanol, isopropanol, butanol, tert-butanol, isobutanol, 3-methyl-1-butanol, 2-methyl-1-butanol, fusel oil, pentanol, hexanol, cyclohexanol, octanol, 2-ethylhexanol, decanol, lauryl alcohol, myristyl alcohol, cetyl alcohol, cetostearyl alcohol, stearyl alcohol, oleyl alcohol, behenyl alcohol, jojoba alcohol, 2-hexadecyl alcohol, 2-octyldodecanol alcohol and isostearyl alcohol.

These amino acid esters may be obtained in particular from natural sources of amino acids. In this case, the amino acids originate from the hydrolysis of natural plant proteins (oat, wheat, soybean, palm or coconut) and then necessarily lead to mixtures of amino acids that subsequently need to be esterified and then N-acylated. The preparation of such amino acids is more particularly described in patent application FR 2 796 550.

The amino acid ester more particularly preferred for its use in the present invention is isopropyl N-lauroylsarcosinate of formula:

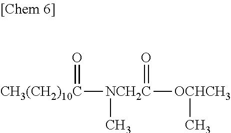

such as the product sold under the name Eldew SL-205 by the company Ajinomoto.

The amino acid esters that are preferably used for the purposes of the present invention, and the synthesis thereof, are described in patent applications EP 1 044 676 and EP 0 928 608 from the company Ajinomoto Co.

The N-acylamino acid ester(s) may be present in the composition according to the invention in a content ranging from 0.1% to 3% by weight, preferably from 0.15% to 1.0%, relative to the total weight of said composition.

A subject of the invention also relates to a composition, in particular a cosmetic composition, preferably in the form of an oil-in-water emulsion comprising:

at least one compound of formula (I) according to the invention

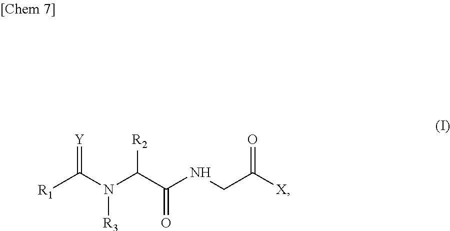

preferably [2-[acetyl-(3-trifluoromethylphenyl)amino]-3-methylbutyrylamino]acetic acid, and

at least one compound of formula (VII) as defined above

R′1(CO)N(R′2)CH(R′3)(CH2)n(CO)OR′4 (VII),

preferably isopropyl N-lauroylsarcosinate.

More particularly, the present invention relates to a composition, in particular a cosmetic composition, in the form of an oil-in-water emulsion comprising:

at least one compound of the family of N-acylamino amides of formula (I) according to the invention;

at least one compound of formula (VII) as defined above

R′1(CO)N(R′2)CH(R′3)(CH2)n(CO)OR′4 (VII),

preferably isopropyl N-lauroylsarcosinate;

at least one alkyl polyglycoside of formula (IV) below:

R₉(O)(G)x (IV)

in which the radical R₉ is a linear or branched C₁₄-C₂₄ alkyl radical;

G represents a reduced sugar comprising from 5 to 6 carbon atoms; and

x denotes a value ranging from 1 to 10, preferably 1 to 4; and optionally

at least one compound chosen from oxyethylenated sorbitan esters and fatty acid esters of a polyoxyalkylene glycol.

The compositions used according to the invention may comprise a physiologically acceptable medium, i.e. a medium that is suitable for the topical administration of a composition, i.e. that is compatible with human keratin materials such as the skin and the nails.

According to the invention, a physiologically acceptable medium is preferentially a cosmetically acceptable medium, i.e. a medium which is free of any odour or unpleasant appearance and which is entirely compatible with the topical administration route.

According to the invention, the term “keratin materials” is intended to mean the skin, of the body, face and/or area around the eyes, the lips, the nails, the mucous membranes, or any other area of bodily skin.

Preferably, the keratin material according to the invention is the skin.

The term “skin” refers to all the skin of the body, and preferably the skin of the face, neckline, neck, arms and forearms, or even more preferably the skin of the face, notably of the forehead, nose, cheeks, chin and area around the eyes.

As specified below, the compound of formula (I) is advantageously present in the compositions in accordance with the invention in a dissolved form.

By way of example, the composition according to the invention may be intended to be administered topically, i.e. by application at the surface of the keratin material under consideration, such as the skin under consideration.

The composition of the invention may be a cosmetic composition (i.e. intended for cosmetic purposes) or a dermatological composition. Preferentially, according to the invention, the composition is a cosmetic composition and even more preferentially a cosmetic composition for topical application.

The term “cosmetic composition” is intended to mean in particular a substance or a preparation intended to be brought into contact with the various superficial parts of the human body, in particular the epidermis, the nails, the lips, with a view, exclusively or mainly, to cleansing them, making them more attractive, fragrancing them, modifying their appearance, protecting them, keeping them in good condition, or correcting body odours.

A subject of the invention is also a process for the cosmetic treatment of keratin materials, comprising the application to the keratin materials, such as the skin, of a composition according to the invention as described above.

In one particular embodiment, a subject of the invention is a cosmetic process for treating ageing, for improving the quality of the barrier function of and/or for regenerating keratin materials, in particular the skin, especially human skin, comprising the application to said keratin materials of a composition as defined above.

More particularly, a subject of the invention is also a cosmetic treatment process for caring for, making up and/or cleansing keratin materials, in particular the skin, comprising the application to said keratin materials, in particular the skin, of a composition according to the invention as described previously.

Said cosmetic treatment process for caring for, making up and/or cleansing the skin is non-therapeutic.

A subject of the invention is also a cosmetic process for preventing and/or treating the signs of ageing of the skin, comprising at least one step of topical application to the keratin materials, such as the skin, of a composition according to the invention as described previously.

The signs of ageing of the skin to be prevented and/or treated in the cosmetic process according to the invention may be chosen from wrinkles and fine lines, and/or for combating wizened, flaccid and/or thinned skin.

A subject of the invention is also the cosmetic use of a composition according to the invention as defined previously, for caring for, making up and/or cleansing keratin materials.

In another embodiment, the invention relates to a cosmetic treatment process for caring for, making up and/or cleansing keratin materials, especially the skin, comprising the topical administration, to an individual, of a composition as defined previously.

The expressions “between . . . and . . . ” and “ranging from . . . to . . . ” or “at least . . . ” or “at least of . . . ” should be understood as being limits inclusive, unless otherwise specified.

The examples below of compositions according to the invention are given as illustrations with no limiting nature. The compounds are indicated as their chemical name or their INCI name.

The contents of the ingredients below are expressed as weight percentage of starting material, unless otherwise indicated.

### Example 1

The following compositions in accordance with the invention were prepared:

TABLE 1

| | Starting | Commercial | | | | |
|---|---|---|---|---|---|---|
| Phase | material | Reference | Composition 3 | Composition 4 | Composition 1 | Composition 2 |
| A | WATER | | 72.3 | 66.894 | 73.19 | 67.47 |
| | SODIUM | CAUSTIC | | 0.206 | 0.11 | 0.03 |
| | HYDROXIDE | LYE(GUJARAT | | | | |
| | | ALKALIES AND | | | | |
| | | CHEMICALS) | | | | |
| | SODIUM | CRISTALHYAL LO | | 0.1 | | 0.5 |
| | HYALURONATE | (SOLIANCE | | | | |
| | | (GIVAUDAN)) | | | | |
| | ACETYL | MEXORYL SAR | 2 | 2 | 1 | 1 |
| | TRIFLUOROMETHYLPHENYL | (CHIMEX) | | | | |
| | VALYLGLYCINE | | | | | |
| | ([2-[acetyl- | | | | | |
| | (3-trifluoro- | | | | | |
| | methylphenyl)amino]- | | | | | |
| | 3-methylbutyrylamino]- | | | | | |
| | acetic acid) | | | | | |
| | PHENOXYETHANOL | SEPICIDE LD | 0.5 | 0.5 | 0.5 | 0.5 |
| | | (SEPPIC) | | | | |
| | DIPROPYLENE | DIPROPYLENEGLYCOL | 2 | | 2 | |
| | GLYCOL | CARE (BASF) | | | | |
| | BUTYLENE | 1,3 BUTYLENE | | 2 | | 2 |
| | GLYCOL | GLYCOL | | | | |
| | | (DAICEL) | | | | |
| | DENAT. | SURFIN | | 2 | | 2 |
| | ALCOHOL | DEHYDRATED | | | | |
| | | ABSOLUTE | | | | |
| | | ALCOHOL | | | | |
| | | 0.1% TBA + | | | | |
| | | 10 MG/L | | | | |
| | | BITREX | | | | |
| | | (TEREOS) | | | | |
| | GLYCERIN | ECOCEROL | 7 | 7 | 7 | 7 |
| | | (ECOGREEN | | | | |
| | | OLEOCHEMICALS) | | | | |
| | PENTYLENE | 616751 | | 2 | | 2 |
| | GLYCOL | HYDROLITE-5 ® | | | | |
| | | (SYMRISE) | | | | |
| | CAPRYL | HYDROLITE CG ® | 0.7 | 0.3 | 0.7 | 0.3 |
| | GLYCOL | (SYMRISE) | | | | |
| B | POLYSORBATE | TWEEN 20- | | 0.2 | | 0.2 |
| | 20 | LQ-(AP) ® | | | | |
| | | from the | | | | |
| | | company Croda | | | | |
| | PEG-30 | CITHROL | 0.5 | | 0.5 | |
| | DIPOLYHYDROXYSTEARATE | DPHS-SO- | | | | |
| | | (MV) ® by the | | | | |
| | | company Croda | | | | |
| | OCTYLDODECANOL | FLUIDANOV | 2 | 2 | 2 | 2 |
| | (and) OCTYLDODECYL | 20 X ® | | | | |
| | XYLOSIDE | (SEPPIC) | | | | |
| | ISOPROPYL LAUROYL | ELDEW SL-205 | | 4.5 | | 4.5 |
| | SARCOSINATE | (AJINOMOTO) | | | | |
| C | CYCLOHEXASILOXANE | XIAMETER PMX-0246 | 6 | | 6 | |
| | | CYCLOHEXASILOXANE(DOW | | | | |
| | | CORNING (DOW | | | | |
| | | CHEMICAL) | | | | |
| | DIMETHICONE | XIAMETER PMX-200 | | 2 | | 2 |
| | | SILICONE FLUID 5 CST | | | | |
| | | (DOW CORNING (DOW | | | | |
| | | CHEMICAL)) | | | | |
| | DIMETHICONE | DOW CORNING EL- | 2 | | 2 | |
| | CROSSPOLYMER | 9240 SILICONE | | | | |
| | | ELASTOMER BLEND | | | | |
| | | (DOW CORNING (DOW | | | | |
| | | CHEMICAL)) | | | | |
| | DIMETHICONE (and) | X-25-7034H (SHIN | | 5 | | 5 |
| | DIMETHICONE/VINYL | ETSU) | | | | |
| | DIMETHICONE | | | | | |
| | CROSSPOLYMER | | | | | |
| D | XANTHAN GUM | RHODICARE | | 0.3 | | 0.3 |
| | | XC (PMC | | | | |
| | | OUVRIE) | | | | |
| | SODIUM | SIMULGEL | 5 | | 5 | |
| | ACRYLATE/SODIUM | EG (SEPPIC) | | | | |
| | ACRYLOYLDIMETHYL | | | | | |
| | TAURATE COPOLYMER | | | | | |
| | (and) ISOHEXADECANE | | | | | |
| | (and) POLYSORBATE 80 | | | | | |
| | POLYACRYLATE | SEPIMAX ZEN | | 1 | | 1 |
| | CROSSPOLYMER-6 | (SEPPIC) | | | | |
| E | TOCOPHERYL | VITAMIN | | | | 0.2 |
| | ACETATE | E ACETATE | | | | |
| | | CARE (BASF) | | | | |
| | BORON NITRIDE | | | 2 | | 2 |

Method for preparing compositions 1, 2, 3 and 4 according to the invention:

Mix phase A and heat to 50° C. to dissolve the active agent.

Add phase B.

Mix and heat the fatty phase C to 30° C.

Incorporate phase C into phase A+B in one step. Mix slowly with a spatula.

Observation of diffusion, mix until an emulsion is obtained.

Mix for a few minutes using a deflocculating device.

Add phase D, then phase E after emulsification.

The compositions according to the invention 1, 2, 3 and 4 can be applied to the skin for cosmetic treatment of the skin, in particular in order to treat the signs of age.

These compositions may be applied regularly to facial skin in order to attenuate the signs of skin ageing.

### Example 2: Evaluation of the Stability

Compositions 1, 2, 3 and 4 of Example 1, in accordance with the invention, were tested for stability according to the following methodology.

Samples of the three formulations were placed in storage for 2 months at 4° C., ambient temperature, 40° C. and/or 45° C. They were then controlled on the basis of the following criteria: macroscopic appearance, microscopic appearance (light microscope magnification ×10, normal and polarized light), pH, viscosity.

It was noted that the three compositions 1, 2, 3 and 4 in accordance with the invention are stable at ambient temperature and at 2 months at 45° C., and exhibit no recrystallization of the compound of formula (I); this compound, [2-[acetyl-(3-trifluoromethylphenyl)amino]-3-methylbutyrylamino]acetic acid, is therefore very well dissolved in these particular combinations.

### Example 3: Evaluation of the Skin Bioavailability

Test for diffusion of the compound of formula (I) through the Strat-M© membrane

This method makes it possible to measure the amount of active agent that diffuses through a membrane which mimics the behaviour of the skin (Strat-M© from Millipore). Each formula is evaluated on 3 to 9 independent Franz cells.

Protocol:

The membrane is placed on a Franz cell, after the receptor compartment has been filled with a phosphate buffer (PBS). The formula containing the active agent is applied to the donor compartment, at a finite dose of 25 mg/cm². The receptor compartment is stirred (600 rpm) for 5 h.

The receptor liquid is sampled after 5 h and the amount of active agent that has diffused is measured (UPLC/UV).

The table below presents the results obtained.

TABLE 2

Bioavailability on Stat-M

| | Composition 1 | Composition 2 |
|---|---|---|
| mean μg/cm2 | 87.56 | 169.67 |
| standard deviation | 35.88 | 4.04 |

Surprisingly, a composition according to the invention comprising at least one alkyl polyglycoside compound of formula (IV), such as octyldodecyl xyloside, combined with an oxyethylenated sorbitan ester, such as polysorbate 20, or a fatty acid ester of a polyoxyalkylene glycol, such as PEG-30 dipolyhydroxystearate, allows very good membrane permeation of a compound of formula (I), such as [2-[acetyl-(3-trifluoromethylphenyl)amino]-3-methylbutyrylamino]acetic acid.

### Test for Bioavailability of a Compound of Formula (I) on Human Skin Ex Vivo

This evaluation is carried out by characterization of the skin absorption of a compound of formula (I) such as [2-[acetyl-(3-trifluoromethylphenyl)amino]-3-methylbutyrylamino]acetic acid.

This skin absorption was evaluated according to OECD Guidelines (OECD guideline for the testing of chemicals: guideline 428, skin absorption: in vitro method (13 Apr. 2004)). The studies are performed on frozen human skin obtained from abdominal plastic surgery following application of a finite dose of formulation (e.g. 5 mg/cm²) for 16 hours. The quantitative analysis is carried by means of a validated LC/MS/MS method, the skin bioavailability being measured according to the definition provided by the SCCS.

A non-parametric statistical analysis based on R-estimators is used.

Table 3 below gives the results obtained.

TABLE 3

Skin bioavailability

| | μg/cm² (±SD) | % Dose applied (±SD) |
|---|---|---|
| Composition 3 | 2.81 ± 1.90 | 3.00 ± 1.88 |
| according to the | | |
| invention | | |
| Composition 4 | 7.16 ± 5.32 | 7.87 ± 5.66 |
| according to the | | |
| invention | | |

Surprisingly, a composition according to the invention comprising at least one alkyl polyglycoside compound of formula (IV), such as octyldodecyl xyloside, combined with an oxyethylenated sorbitan ester, such as polysorbate 20, or a fatty acid ester of a polyoxyalkylene glycol, such as PEG-30 dipolyhydroxystearate, allows good skin bioavailability of a compound of formula (I), such as [2-[acetyl-(3-trifluoromethylphenyl)amino]-3-methylbutyrylamino]acetic acid.

Taking these results into account along with those presented above in regard to stability shows that the compositions in accordance with the invention are able to jointly have excellent stability and a probably greater efficacy of [2-[acetyl-(3-trifluoromethylphenyl)amino]-3-methylbutyrylamino]acetic acid given the increase in skin bioavailability observed.

1. A composition comprising:
at least one alkyl polyglycoside of formula (IV) below:
R₉(O)(G)ₓ (IV)
in which the radical R₉ is a linear or branched C₁₄-C₂₄ alkyl radical;
G represents a reduced sugar comprising from 5 to 6 carbon atoms; and
x denotes a value ranging from 1 to 10; and
at least one compound of formula (I) below: 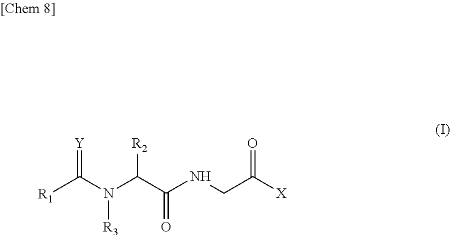
in which:
the radical Y represents O or S,
the radical R1 represents:
(i) a hydrogen atom;
(ii) a linear, branched or cyclic, saturated or unsaturated hydrocarbon-based radical comprising 1 to 18 carbon atoms,
optionally substituted with 1 to 5 groups, which may be identical or different, chosen from —OH; —OR; —O—COR; —SH; —SR; —S—COR; —NH₂; -—NHR; —NRR′; —NH—COR; -Hal (halogen); —CN; —COOR; —COR; —P(O)—(OR)₂; —SO₂—OR; with R and R′ representing, independently of one another, a linear, branched or cyclic, saturated or unsaturated hydrocarbon-based radical comprising 1 to 6 carbon atoms, which is optionally halogenated, or even perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
(iii) a radical chosen from the radicals —OR; —NH₂; —NHR; —NRR′; —NH—COR; —COOR; —COR; with R and R′ representing, independently of one another, a linear, branched or cyclic, saturated or unsaturated hydrocarbon-based radical comprising 1 to 6 carbon atoms, which is optionally halogenated or perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
the radical R2 represents a linear, branched or cyclic, saturated or unsaturated hydrocarbon-based group comprising 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen); —CN; —COOR; —COR;
with R and R′ representing, independently of one another, a linear, branched or cyclic, saturated or unsaturated hydrocarbon-based radical comprising 1 to 6 carbon atoms, which is optionally halogenated or perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
the radical R3 represents a radical chosen from those of formula (II) or (III):
(II) —A—C6H(5-y)-By
(III) —C6H(5-y′)-By
in which:
y is an integer between 0 and 5 included, and y′ is an integer between 1 and 5 included;
A is a linear or branched, saturated or unsaturated, divalent hydrocarbon-based radical comprising 1 to 18 carbon atoms,
optionally substituted with 1 to 5 groups, which may be identical or different, chosen from —OH;
—OR; —O—COR; —SH; —SR; —S—COR; —NH₂; —NHR; —NRR′; —NH—COR; -Hal (halogen or perhalogen); —CN; —COOR; —COR; -—NO₂; —SO₂—OR;
with R and R′ representing, independently of one another, a linear, branched or cyclic, saturated or unsaturated hydrocarbon-based radical comprising 1 to 6 carbon atoms, which is optionally halogenated or perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
B is a linear or branched, saturated or unsaturated hydrocarbon-based radical comprising 1 to 18 carbon atoms,
optionally substituted with 1 to 5 groups, which may be identical or different, chosen from —OH; —OR; —O—COR; —SH; —SR; —S—COR; —NH₂; —NHR; —NRR′; —NH—COR; -Hal (halogen or perhalogen); —CN; —COOR; —COR; —NO₂; —SO₂—OR;
with R and R′ representing, independently of one another, a linear, branched or cyclic, saturated or unsaturated hydrocarbon-based radical comprising 1 to 6 carbon atoms, which is optionally halogenated or perhalogenated,
it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
the radical X represents a radical chosen from —OH, —OR4, —NH₂, —NHR4, —NR4R5, —SR4, ——COOR4; —COR4;
with R4 and R5 representing, independently of one another, a linear, branched or cyclic, saturated or unsaturated hydrocarbon-based radical comprising 1 to 6 carbon atoms, optionally substituted with 1 to 5 groups, which may be identical or different, chosen from —OH; —OR; —O—COR; —SH; —SR; —S—COR; —NH₂; —NHR; —NRR′; —NH—COR; -Hal (halogen or even perhalogen); —CN; —COOR; —COR; with R and R′ representing, independently of one another, a linear, branched or cyclic, saturated or unsaturated hydrocarbon-based radical comprising 1 to 6 carbon atoms, which is optionally halogenated or perhalogenated; it being possible for said radicals R and R′ to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
it being possible for said radicals R4 and R5 to form, together with N, a 5 or 6-membered carbon-based ring which may also comprise at least one heteroatom chosen from O, N and/or S in the ring, and/or to be substituted with 1 to 5 groups, which may be identical or different, chosen from —OH; —OR″; —O—COR″; —SH; —SR″; —S—COR″; —NH₂; —NHR″; —NH—COR″; -Hal (halogen); —CN; —COOR″; —COR″; with R″ representing a linear, branched or cyclic, saturated or unsaturated hydrocarbon-based radical comprising 1 to 6 carbon atoms, which is optionally halogenated or perhalogenated;
inorganic or organic acid salts thereof, optical isomers thereof, in isolated form or as a racemic mixture.

2. The composition according to claim 1 1, which is a composition in the form of an oil-in-water emulsion.

3. The composition according to claim 1, wherein said composition also comprises at least one additional emulsifying surfactant.

4. The composition according to claim 1, wherein, for said compound of formula (I):
the radical Y represents oxygen; and/or
the radical R1 represents hydrogen or an optionally substituted linear or branched, saturated or unsaturated hydrocarbon-based radical comprising 1 to 12 carbon atoms; and/or
the substituents of R1 are chosen from —OH, —OR and/or —P(O)—-(OR)₂ with R representing a linear, branched or cyclic, saturated or unsaturated hydrocarbon-based radical comprising 1 to 6 carbon atoms, which is optionally halogenated or perhalogenated; and/or
the radical R2 represents an optionally substituted linear, branched or cyclic, saturated or unsaturated hydrocarbon-based radical comprising 1 to 12 carbon atoms; and/or
the substituents of R2 are chosen from —OH and —OR with R representing a linear, branched or cyclic, saturated or unsaturated hydrocarbon-based radical comprising 1 to 6 carbon atoms, which is optionally halogenated or perhalogenated; and/or
the radical R3 represents a radical of formula —C₆H(5-y′)-By′ for which y′=1, 2 or 3; or a radical of formula —A—C₆H(5-y)-By for which y=0, 1 or 2; and/or
the radical A of R3 is an optionally substituted linear or branched, saturated or unsaturated, divalent hydrocarbon-based radical comprising 1 to 12 carbon atoms; and/or
the radical B of R3 is an optionally substituted linear or branched, saturated or unsaturated hydrocarbon-based radical comprising 1 to 12 carbon atoms; and/or
the substituents of A and/or of B are chosen from -Hal (halogen, or perhalogen); —CN; —COOR; -—NO₂; —SO₂—OR; with R representing a linear, branched or cyclic, saturated or unsaturated hydrocarbon-based radical comprising 1 to 6 carbon atoms, which is optionally halogenated or perhalogenated; and/or
the radical X represents a radical chosen from —OH or —OR4 with R4 representing an optionally substituted linear, cyclic or branched, saturated or unsaturated hydrocarbon-based radical comprising 1 to 6 carbon atoms; and/or
the substituents of R4 of X are chosen from —OH and —OR with R representing a linear, branched or cyclic, saturated or unsaturated hydrocarbon-based radical comprising 1 to 6 carbon atoms, which is optionally halogenated or perhalogenated.

5. The composition according to claim 1, wherein, for said compound of formula (I):
the radical R1 represents a methyl, ethyl, propyl or isopropyl radical, optionally substituted with an —OH for —P(O)—(OR)₂ group with R representing methyl, ethyl, propyl or isopropyl; and/or
the radical R2 represents a methyl, ethyl, propyl, isopropyl, n-butyl, ter-butyl or isobutyl radical; and/or
the radical R3 represents a group chosen from one of the formulae below: 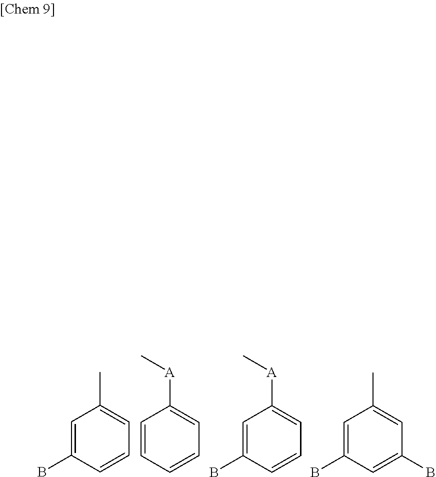
in which the divalent radical A is a methylene, an ethylene, a propylene and/or the radical B is a methyl, ethyl, propyl or isopropyl radical, substituted with one or more halogens;
the radical X represents a radical chosen from —OH, —OCH₃, —OC₂H₅, —O—C₃H₇ or —OC₄H₉.

6. The composition according to claim 1, wherein, said compound of formula (I) is chosen from:
[2-[acetyl-(3-trifluoromethylphenyl)amino]-3-methylbutyrylamino]acetic acid,
ethyl[2-[acetyl-(3-trifluoromethylphenyl)amino]-3-methylbutyrylamino]acetate,
[2-(acetylbenzylamino)-3-methylbutyrylamino]acetic acid,
ethyl[2-(acetylbenzylamino)-3-methylbutyrylamino]acetate, and
ethyl(2-[benzyl[(diethoxyphosphoryl)acetylamino]-3-methylbutyrylamino)acetate.

7. The composition according to claim 1, wherein the alkyl polyglycoside(s) are chosen from the compounds of formula (IV′) below:
R₉′—O-G (IV′)
in which:
the radical R₉′ is a branched alkyl radical comprising from 14 to 24 carbon atoms and G denotes a reduced sugar comprising from 5 to 6 carbon atoms.

8. The composition according to claim 1, also comprising at least one fatty alcohol comprising from 14 to 24 carbon atoms and having the same fatty chain as that of the alkyl polyglycoside.

9. The composition according to claim 8, in which the alkyl polyglycoside is octyldodecyl xyloside and the fatty alcohol is octyldodecanol.

10. The composition according to claim 8, in which the mixture of octyldodecyl xyloside and octyldodecanol is present in a content ranging from 0.01% to 50% by weight relative to the total weight of the composition.

11. The composition according to claim 9, in which the amount of octyldodecyl xyloside ranges from 20% to 30% by weight relative to the total weight of the mixture of octyldodecyl xyloside and octyldodecanol.

12. The composition according to claim 9, in which the amount of octyldodecanol ranges from 70% to 80% by weight relative to the total weight of the mixture of octyldodecyl xyloside and octyldodecanol.

13. The composition according to claim 1, wherein said compound(s) of formula (I) is (are) present in a content ranging from 0.001% to 50% by weight relative to the total weight of the composition.

14. The composition according to claim 3, wherein the oxyethylenated sorbitan ester(s) is(are) chosen from C₈-C₃₀ fatty acid monoesters and polyesters of sorbitan, having from 1 to 50 ethylene oxide units.

15. The composition according to claim 3, wherein the fatty acid ester(s) of a polyoxyalkylene glycol is (are) chosen from polyhydroxylated fatty acid esters of polyethylene glycol, in particular in which the fatty acid comprises from 12 to 20 carbon atoms and the polyethylene glycol comprises from 4 to 5 mol of ethylene oxide.

16. The composition according to claim 15, also comprising at least one N-acylamino acid ester of formula (VII) below:
R′₁(CO)N(R′₂)CH(R′₃)(CH₂)n(CO)OR′₄ (VII)
in which:
n is an integer equal to 0, 1 or 2,
R′₁ represents a linear or branched C₅-C₂₁ alkyl or alkenyl radical,
R′₂ represents a hydrogen atom or a C₁ to C₃ alkyl group,
R′₃ represents a radical chosen from the group formed by a hydrogen atom, a methyl group, an ethyl group and a linear or branched C₃ or C₄ alkyl radical,
R′₄ represents a linear or branched C₁ to C₁₀ alkyl or C₂ to C₁₀ alkenyl radical, or a sterol residue.

17. The composition according to claim 16, in which the N-acylamino acid ester of formula (VII) is isopropyl N-lauroylsarcosinate of formula:

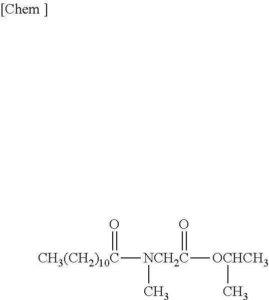

18. A cosmetic process for treating keratin materials, comprising the application to the keratin materials of a composition as claimed in claim 1.

19. A cosmetic process for treating ageing, for improving the quality of the barrier function of and/or for regenerating keratin materials comprising the application to said keratin materials of a composition as defined according to claim 1.

20. A cosmetic process for caring for, protecting and/or making up of bodily and/or facial skin.
